(19) United States
(12) Patent Application Publication
(10) Pub. No.: US 2006/0029657 A1
(43) Pub. Date: Feb. 9, 2006

(54) Topical skin protectant compositions

(75) Inventors: Karl F. Popp, Schodack Landing, NY (US); John A. Wagner, Suwanee, GA (US)

Correspondence Address: NATH & ASSOCIATES, 112 South West Street, Alexandria, VA, 22314, US

(73) Assignee: Stiefel Laboratories, Inc.

(21) Appl. No.: 11/089,030

(22) Filed: Mar. 25, 2005

Related U.S. Application Data
(60) Provisional application No. 60/556,428, filed on Mar. 26, 2004.

(51) Int. Cl. A61K 9/127; A61K 8/73

(52) U.S. Cl. 424/450; 424/70.13

(57) ABSTRACT

Skin protectant compositions that are free of cholesterol and suitable for topical application to skin of a mammal. In a preferred embodiment, these skin protectant compositions comprise a ceramide; a squalane; a phytosterol-containing liposome; a phospholipid-containing ingredient; at least one triglyceride; and at least one dermatologically acceptable excipient. These compositions are capable of restoring or repairing a skin lipid barrier of a mammal, and treating skin conditions associated therewith.

This application claims priority to U.S. Provisional Patent Application Ser. No. 60/556,428, filed on Mar. 26, 2004, the contents of which are hereby incorporated by reference in their entirety.

### FIELD OF THE INVENTION

The present subject matter relates generally to skin protectant compositions that are free of cholesterol and suitable for topical application to skin of a mammal. In a preferred embodiment, these skin protectant compositions comprise a ceramide; a squalane; a phytosterol-containing liposome; a phospholipid-containing ingredient; at least one triglyceride; and at least one dermatologically acceptable excipient. These compositions are capable of restoring or repairing a skin lipid barrier of a mammal, and treating skin conditions associated therewith.

### BACKGROUND OF THE INVENTION

The skin is the largest organ of the body and serves as a barrier protecting mammalian organisms from both aqueous and xerotic ambient environments. The maintenance of this barrier against excessive transcutaneous water loss to the environment is critical to survival of all terrestrial animals. In mammals, this barrier is formed by the anucleate, cornified, outermost layers of the epidermis, collectively known as the stratum corneum.

Both the surfaces of mucous membranes and the deepest layers of the stratum corneum contain high concentrations of glycosphingolipids which are typically metabolized progressively to ceramides as the stratum granulosum becomes anucleate with outward maturation. However, localized or generalized perturbations of the epidermal barrier, such as those that occur in a variety of diseases and conditions of the skin and mucous membrane, can interfere with these processes. These perturbations not only contribute significantly to the morphology of the cutaneous lesions, but also activate certain skin diseases, for example the Koebner phenomenon in psoriasis.

In this regard, substances which effect barrier repair are typically additionally effective as moisturizers. However, while all effective moisturizers will temporarily decrease visible scaling and roughness, they usually offer little or no improvement to the integrity of the stratum corneum barrier. In fact, common moisturizers and emollients can cause disruptions of the barrier function.

For example, while scaling and roughness of the skin are manifestations of an abnormally desquamating stratum corneum, these conditions often do not correlate with the function of the stratum corneum barrier. Patients suffering from atopic dermatitis, for example, have skin with an incompetent barrier, as measured by trans-epidermal water loss, but no visible scaling.

Moisturizers are defined as substances which increase the stratum corneum water content. Skin conductance measurements are known as the most accurate assay of the water content. High skin conductance measurements indicate high water content. While high water content indicates a high degree of moisturization, it is not an indication of good barrier function. Mucous membranes, for example, are moist with an extremely high water content but typically exhibit a poor barrier function. Similarly, the thickness of the stratum corneum typically does not correlate with barrier function. For example, palms and soles, which appear normal, typically have the thickest stratum corneum and a high water content but relatively poor barrier function.

Instead, intercellular, lamellar, bilayer sheets of stratum corneum lipids are usually the key constituents of a functional skin barrier. These epidermal lipids are a mixture of polar and nonpolar species. The most dominant lipids by weight are ceramides (40%), cholesterol (20-25%) and free fatty acids (20-25%). These fatty acids include essential fatty acids, such as linoleic acid, as well as additional nonessential fatty acids. The human epidermis further contains a unique acylsphingolipid whose molecular structure includes a sphingoid backbone with a 30-carbon, α-hydroxy acid residue joined to the backbone through an amide linkage, the residue itself being ω-esterified with linoleic acid.

Although each of these lipid species is important for stratum corneum homeostasis, ceramides are of particular importance because of their large weight contribution and structural characteristics. The moisturization properties of ceramides are known. For example, Japanese Published Patent Application No. 24391-1987 to Kao Company Limited discloses a formulation containing bovine ceramide with a sphingoid base of 10 to 26 carbon atom length and one or more of any other stratum corneum lipids. The purpose of this formulation is to increase water retention and improve skin roughness (moisturization).

Despite the potential efficacy of such lipids as moisturizer ingredients, studies have demonstrated that many individual lipids actually impede rather than facilitate barrier repair when applied to damaged skin. For example, in mice which suffer a barrier defect due to a deficiency in the essential fatty acid, linoleic acid, topical application of linoleic acid alone actually further aggravates barrier dysfunction until the systemic deficiency state is corrected.

Administration of the individual lipids to damaged skin, then, will likely worsen skin lesions and mucous membrane diseases. Two-component lipid systems similarly do not accelerate barrier recovery when applied to damaged skin. One possible explanation for this negative action is that an acute or chronically damaged epidermal barrier responds differently than either normal skin or merely dry, rough skin when epidermal lipids are applied.

However, the importance of the major epidermal lipids, taken in combination, to the integrity of the epidermal barrier is demonstrated by their increased synthesis after both acute and chronic barrier disruption. Inhibition of their respective rate-limiting enzymes typically causes a reduction in concentration of any one of these three major epidermal lipids. This often produces significant delay in the recovery of barrier function, i.e. the prevention of water loss from the skin.

Numerous tests have been conducted to determine the critical aspects of the major epidermal lipids on barrier function. For example, lovastatin, an inhibitor of cholesterol synthesis, produces a barrier defect when applied to normal epidermis. After lovastatin is applied, cholesterol synthesis rapidly normalizes, but fatty acid synthesis remains elevated. This suggests that a disturbance in the fatty acid to cholesterol ratio accounts for the perturbed barrier function.

Numerous cutaneous conditions have been shown to involve or give rise to such a disrupted or dysfunctional epidermal barrier or barrier function, including:

1) Causes of morbidity and mortality in premature infants less than 33 weeks of gestational age, such as fluid and electrolyte abnormalities, hypothermia, and infection with the skin being the portal of entry. The development of mature barrier function coincides with the deposition of adequate amounts of the major epidermal lipids in appropriate proportions.

2) Eczematous dermatitides, which are a group of inflammatory hyperproliferative skin diseases characterized by poorly demarcated, scaly, itchy or tender patches that may involve wide-spread areas of the body. Two of the most common types are atopic and seborrheic dermatitis. Both have a genetic predisposition and display abnormalities of stratum corneum lipids and barrier function even in clinically uninvolved skin. The other major eczematous dermatitides usually result from environmental or occupational insults of solvents, chemicals, detergents, hot water, low ambient humidity, or ultraviolet or X radiation. These other disorders include allergic or irritant contact dermatitis, eczema craquelee, photoallergic, phototoxic, or phytophotodermatitis, radiation, and stasis dermatitis. Eczema craquelee begins as dehydrated or dry skin that reaches such severity that complete destruction of the epidermal barrier occurs, which results in inflammation and hyperproliferation.

3) Ulcers and erosions resulting from trauma or ischemia of the skin or mucous membranes. These insults include chemical or thermal burns, and vascular compromise as in venous, arterial, embolic or diabetic ulcers. The lesions are not only painful but form a portal for pathogenic microbes.

4) Ichthyoses common to rare genetic diseases characterized by disorders of abnormal epidermal cornification with or without associated abnormal barrier function and epidermal hyperproliferation.

5) Epidermolysis bullosae, which are a group of rare genetic diseases resulting from an absence or defect in epidermal/dermal cohesion. Cutaneous trauma to normal skin with normal daily activity results in complete or partial loss of the epidermis, often producing blisters, erosions, and ulcers.

6) Psoriasis, which is a markedly hyperproliferative, inflammatory papulosquamous disease typically characterized by sharply demarcated, scaly plaques most frequently located at areas of the body which suffer trauma, specifically knees, elbows, hands, feet, and scalp.

7) Cutaneous changes of intrinsic aging and photoaging (dermatoheliosis) resulting from environmental ravages combined with intrinsic changes which can produce atrophy, fragility, inelasticity, decreased cell cohesion, hypoproliferation, and delayed healing after insults to the barrier. The stratum corneum lipids display a depletion of ceramide and nonpolar lipid species with a relative increase in cholesterol.

8) Limiting factors for occupational or athletic performance, even for the occasional recreational athlete, such as frictional blistering of the skin by mechanical shearing forces.

9) Limiting factors for the topical use of corticosteroids, especially in the young and elderly, such as cutaneous atrophy which can predispose to infection and slow the rate of healing.

Common therapies used for many of these disorders include topical corticosteroids, systemic antihistamines with or without antibiotics, antibiotics, occlusive dressings, vascular compression bandages, systemic and topical retinoids, topical α-hydroxy and salicylic acids, diphenylhydantoin, sulfones, antineoplastic agents, anthralin, tar, psoralens and ultraviolet A or B light. Unfortunately, some of these treatments can at times produce significant adverse side effects, such as topical irritation, serious systemic and/or cutaneous toxicity, and other severe systemic side effects.

Moreover, these treatments at times are followed by a rapid rebound of the disease when they are withdrawn. In fact, rather than repair the skin barrier, some of these treatments actually worsen it. Further, the skin can remain excessively sensitive for months after the apparent clinical resolution of the lesions using some of these treatments. This results in rapid rebound of the lesions with significantly less environmental insult. Therefore, there is a great need for an effective therapeutic formulation that will normalize the barrier of both clinically uninvolved and involved skin to prevent disease exacerbations and/or limit disease extent.

U.S. Pat. No. 5,643,899 discloses potential compositions directed specifically to treatment of epidermal barrier disorders such as hyperproliferative cutaneous diseases, papulosquamous diseases, and eczematous diseases. The disclosed compositions contain various combinations of essential lipids that must include cholesterol and a ceramide, particularly acylceramide. However, the compositions disclosed in this patent are not capable of affecting the epidermal barrier for an extended period of time. Accordingly, it is necessary to frequently apply the compositions to achieve effective skin barrier repair.

In this regard, U.S. Pat. No. 5,508,034 discloses other potential compositions containing various lipids naturally found in the stratum corneum as essential components for the treatment of dry skin disorders. These compositions must contain a fatty acid, cholesterol, and a phospholipid or a glycolipid. Accordingly, these compositions similarly are unable to affect the epidermal barrier for an extended period of time.

Accordingly, lipid-containing compositions designed specifically for the effective treatment of epidermal barrier disorders were previously unknown in the art. Further, the previously known compositions were not contemplated as capable of providing an extended period of action on the epidermal barrier.

For these reasons, there remains a need for lipid compositions that are effective in treating a skin lipid barrier generally, or hyperproliferative cutaneous diseases specifically, over an extended period of time. There further remains a need for compositions comprising any of the known or potential therapeutic compounds whose utility have been prevented and/or compromised due to cutaneous irritation or barrier disruption. Such compositions should overcome certain formulation, stability, and duration of effectiveness problems which have been associated with the prior compositions, and provide improved compositions which are less irritating, easy to formulate, have a smooth consistency after formulation, are substantially uniform, are adequately stable, and have a sufficiently long storage life. The present subject matter addresses these needs.

### SUMMARY OF THE INVENTION

The present subject matter relates to a delivery system for topical application to skin of a mammal comprising:

a therapeutically effective amount of a skin protectant composition comprising:
a) a ceramide,
b) a squalane,
c) a phytosterol-containing liposome,
d) a phospholipid-containing ingredient,
e) at least one triglyceride, and
f) at least one dermatologically acceptable excipient; and

a carrier for said skin protectant composition, wherein said skin protectant composition is free of cholesterol.

In a preferred embodiment, the present subject matter relates to a method for restoring or repairing a skin lipid barrier of a mammal comprising:

topically applying to skin of a mammal in need thereof a therapeutically effective amount of a composition comprising:

a) a ceramide;

b) a squalane;

c) a phytosterol-containing liposome;

d) a phospholipid-containing ingredient;

e) at least one triglyceride; and

f) at least one dermatologically acceptable excipient,

wherein said composition increases intercellular adhesion in said skin to restore or repair said skin lipid barrier and enhance moisturization of said skin, and wherein said composition is free of cholesterol.

In another preferred embodiment, the present subject matter relates to a process for manufacturing a composition suitable for topical administration comprising an oil-in-water emulsion, said process comprising:

1) providing a change in flow of an aqueous phase and an oil phase comprising a squalane, a phytosterol-containing liposome, and at least one triglyceride to provide an oil-in-water emulsion;

2) adding a ceramide and a phospholipid-containing ingredient to said emulsion; and

3) recovering a topical composition.

In still another preferred embodiment, the present subject matter relates to a method for treating a skin condition in a mammal having sensitive skin with an extended release formulation comprising:

topically applying to skin of a mammal in need thereof a therapeutically effective extended release amount of a composition comprising:

a) a ceramide;

b) a squalane;

c) a phytosterol-containing liposome;

d) a phospholipid-containing ingredient;

e) at least one triglyceride; and

f) at least one dermatologically acceptable excipient,

wherein said composition provides an extended release of said ceramide, squalane, phytosterol-containing liposome, phospholipids-containing ingredient, and triglyceride sufficient to treat said sensitive skin without irritating said sensitive skin, and wherein said composition is free of cholesterol.

In yet another preferred embodiment, the present subject matter relates to a method for treating a skin condition in a human child having sensitive skin with an extended release formulation comprising:

topically applying to skin of a human child in need thereof a therapeutically effective extended release amount of a composition comprising:

a) a ceramide;

b) a squalane;

c) a phytosterol-containing liposome;

d) a phospholipid-containing ingredient;

e) at least one triglyceride; and

f) at least one dermatologically acceptable excipient,

wherein said composition provides an extended release of said ceramide, squalane, phytosterol-containing liposome, phospholipids-containing ingredient, and triglyceride sufficient to treat said sensitive skin without irritating said sensitive skin, and wherein said composition is free of cholesterol.

In still yet another preferred embodiment, the present subject matter relates to a method for reducing manifestations of dry skin while enhancing skin repair in a mammal comprising:

daily topically applying to skin of a mammal in need thereof a therapeutically effective amount of a composition comprising:

a) a ceramide;

b) a squalane;

c) a phytosterol-containing liposome;

d) a phospholipid-containing ingredient;

e) at least one triglyceride; and

f) at least one dermatologically acceptable excipient,

wherein said composition increases intercellular adhesion in said skin resulting in said reducing manifestations of dry skin and said enhancing skin repair, and wherein said composition is free of cholesterol.

In a further preferred embodiment, the present subject matter relates to a method for improving skin barrier function of a mammal comprising:

topically applying to skin of a mammal in need thereof a therapeutically effective amount of a composition having a pH of from about 3 to about 9 comprising:

a) a ceramide;

b) a squalane;

c) a phytosterol-containing liposome;

d) a phospholipid-containing ingredient;

e) at least one triglyceride; and

f) at least one dermatologically acceptable excipient,

wherein said composition normalizes the pH of said skin resulting in said improved skin barrier function, and wherein said composition is free of cholesterol.

In yet another preferred embodiment, the present subject matter relates to a process for preparing a composition suitable for topical administration comprising an oil-in-water emulsion, said process comprising:

1) preparing an aqueous phase comprising about 5 to about 20% of the overall weight of the composition of at least one moisturizer, a first gelling agent, and about 10 to about 60% of the overall weight of the composition of water,

2) cooling said aqueous phase to a temperature of about 40 to about 50° C.;

3) preparing an oil phase comprising about 0.1 to about 5% by weight of the overall weight of the composition of a squalane, about 0.1 to about 5% by weight of the overall weight of the composition of a phytosterol-containing liposome, about 5 to about 25% of the overall weight of the composition of at least one triglyceride, and a second gelling agent;

4) adding said water phase to said aqueous phase while stirring at a temperature of about 40 to about 50° C. to obtain an emulsion;

5) cooling said emulsion to a temperature of about 25 to about 35° C.;

6) adding a ceramide and a phospholipid-containing ingredient to said emulsion; and

7) recovering a topical composition.

In a further preferred embodiment, the present subject matter relates to a process for manufacturing a composition suitable for topical administration comprising an oil-in-water emulsion, said process comprising:

1) providing a change in flow of an aqueous phase and an oil phase to provide an oil-in-water emulsion;

2) adding a ceramide and a phospholipid-containing ingredient to said emulsion; and

3) recovering a topical composition,

wherein said aqueous phase comprises about 5 to about 20% of the overall weight of the composition of at least one moisturizer, a first gelling agent, and about 10 to about 60% of the overall weight of the composition of water,

and wherein said oil phase comprises about 0.1 to about 5% by weight of the overall weight of the composition of a squalane, about 0.1 to about 5% by weight of the overall weight of the composition of a phytosterol-containing liposome, about 5 to about 25% of the overall weight of the composition of at least one triglyceride, and a second gelling agent.

In still another further preferred embodiment, the present subject matter relates to a process for preparing a composition suitable for topical administration comprising an oil-in-water emulsion that reduces dependence on emulsifiers, said process comprising:

1) preparing an aqueous phase comprising about 5 to about 20% of the overall weight of the composition of at least one moisturizer, a first gelling agent, and about 10 to about 60% of the overall weight of the composition of water,

2) cooling said aqueous phase to a temperature of about 40 to about 50° C.;

3) preparing an oil phase comprising about 0.1 to about 5% by weight of the overall weight of the composition of a squalane, about 0.1 to about 5% by weight of the overall weight of the composition of a phytosterol-containing liposome, about 5 to about 25% of the overall weight of the composition of at least one triglyceride, and a second gelling agent;

4) adding said water phase to said aqueous phase while stirring at a temperature of about 40 to about 50° C. to obtain an emulsion;

5) cooling said emulsion to a temperature of about 25 to about 35° C.;

6) adding a ceramide and a phospholipid-containing ingredient to said emulsion; and

7) recovering a topical composition.

In still yet another preferred embodiment, the present subject matter relates to a skin protectant composition suitable for topical application to skin of a mammal comprising:

a) about 0.001 to about 1.5% by weight of a ceramide;

b) about 0.1 to about 5% by weight of a squalane;

c) about 0.2 to about 5% by weight of a phytosterol-containing liposome;

d) about 0.5 to about 5% by weight of a phospholipid-containing ingredient;

e) about 8 to about 30% by weight of at least one triglyceride; and

f) at least one dermatologically acceptable excipient,

wherein said skin protectant composition is free of cholesterol.

### DETAILED DESCRIPTION OF THE INVENTION

### Definitions

The term “administering” as used herein refers to any method which, in sound medical or cosmetic practice, delivers the composition to a subject in such a manner so as to provide a positive effect on a dermatological disorder, condition, or appearance. The compositions are preferably administered such that they cover the entire area to be treated.

As used herein, an “aerosol” is a pressurized dosage form which upon actuation emits a dispersion of liquid and/or solid materials in a gaseous medium. The dosage form is packaged under pressure in a suitable container equipped with a valve assembly. When the valve is opened, the internal pressure forces the aerosol out the valve. The “aerosol” dosage form described herein is synonymous with a “foam” dosage form, referring to a coarse dispersion of gas in liquid in which the volume of the gas is considerably larger than that of the liquid.

As used herein, a “buffer” or “buffering agent” refers to a specific pH adjusting agent added to a composition to convey a certain designated pH to the composition. The present compositions do not require the addition of such a specific designated buffer or buffering agent to maintain a particular designated pH. Rather, the present compositions are uniquely formulated so that they are inherently buffered without the need for a specific buffering agent.

As used herein, the phrase “degradation products” refers to the product(s) produced by decomposition of one or more of the functional ingredients of the compositions used according to the present methods.

As used herein, the phrases an “effective amount” or a “therapeutically effective amount” refers to an amount of a composition or component thereof sufficient enough to have a positive effect on the area of application. Accordingly, these amounts are sufficient to modify the skin disorder, condition, or appearance to be treated but low enough to avoid serious side effects, within the scope of sound medical advice. A therapeutically effective amount will cause a substantial relief of symptoms when applied repeatedly over time. Effective amounts will vary with the particular condition or conditions being treated, the severity of the condition, the duration of the treatment, the specific components of the composition being used, and like factors.

As used herein, an “extended release” refers to a release rate that is different from the normal release rate of designated components. Accordingly, this term indicates that the release rates of any of the designated ingredients in the present compositions have been modified to achieve a delayed, sustained, controlled, and/or extended release in comparison to the normal release rate of these ingredients. Similarly, the present compositions are capable of providing extended beneficial effects on an area to which they are applied. For example, the present compositions are capable of providing an extended moisturization in that they provide a delayed moisturization of the skin (i.e. the moisturization commences later in time after administration than with administration of a previous product) as well as an extended moisturization of the skin (i.e. the moisturization continues for a longer period of time after administration than with administration of a previous product) in comparison with previous products.

As used herein, the phrase an “extended period of time” refers to the shelf life of a composition used according to the present methods, including time spent on the shelf at a pharmacy as well as the entire time period after sale of the composition during which the composition remains effective for the indicated use.

As used herein, the term a “liposome” refers to a completely closed bilayer membrane containing an encapsulated aqueous phase. Liposomes may be any variety of multilamellar vesicles or unilamellar vesicles or structures. Usually, the liposome bilayer membrane has a structure such that the hydrophobic “tails” of a lipid contained in the liposome orient toward the center of the bilayer while the hydrophobic “heads” orient towards the aqueous phase. Methods for the formation of sterol containing liposomes are described in U.S. Pat. No. 6,352,716, the entire contents of which are hereby incorporated by reference.

As used herein, the phrase “pharmaceutically acceptable salts” refers to salts of certain ingredient(s) which possess the same activity as the unmodified compound(s) and which are neither biologically nor otherwise undesirable. A salt can be formed with, for example, organic or inorganic acids. Non-limiting examples of suitable acids include acetic acid, acetylsalicylic acid, adipic acid, alginic acid, ascorbic acid, aspartic acid, benzoic acid, benzenesulfonic acid, bisulfic acid, boric acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, glucoheptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthylanesulfonic acid, naphthylic acid, nicotinic acid, nitrous acid, oxalic acid, pelargonic, phosphoric acid, propionic acid, saccharin, salicylic acid, sorbic acid, succinic acid, sulfuric acid, tartaric acid, thiocyanic acid, thioglycolic acid, thiosulfuric acid, tosylic acid, undecylenic acid, and naturally and synthetically derived amino acids.

If organic bases are used, poorly volatile bases are preferably employed, for example low molecular weight alkanolamines such as ethanolamine, diethanolamine, N-ethylethanolamine, N-methyldiethanolamine, triethanolamine, diethylaminoethanol, 2-amino-2-methyl-n-propanol, dimethylaminopropanol, 2-amino-2-methylpropanediol, and triisopropanolamine. Ethanolamine is particularly preferred in this regard. Further poorly volatile bases which may be mentioned are, for example, ethylenediamine, hexamethylenediamine, morpholine, piperidine, piperazine, cyclohexylamine, tributylamine, dodecylamine, N,N-dimethyldodecylamine, stearylamine, oleylamine, benzylamine, dibenzylamine, N-ethylbenzylamine, dimethylstearylamine, N-methylmorpholine, N-methylpiperazine, 4-methylcyclohexylamine, and N-hydroxyethylmorpholine.

Salts of quaternary ammonium hydroxides such as trimethylbenzylammonium hydroxide, tetramethylammonium hydroxide, or tetraethylammonium hydroxide can also by used, as can guanidine and its derivatives, in particular its alkylation products. However, it is also possible to employ as salt-forming agents, for example, low molecular weight alkylamines such as methylamine, ethylamine, or triethylamine. Suitable salts for the components to be employed according to the present subject matter are also those with inorganic cations, for example alkali metal salts, in particular sodium, potassium, or ammonium salts, alkaline earth metal salts such as, in particular, the magnesium or calcium salts, as well as salts with bi- or tetravalent cations, for example the zinc, aluminum, or zirconium salts. Also contemplated are salts with organic bases, such as dicyclohexylamine salts; methyl-D-glucamine; and salts with amino acids, such as arginine, lysine, and so forth. Also, the basic nitrogen-containing groups can be quaternized with such agents as lower alkyl halides, such as methyl, ethyl, propyl, and butyl chlorides, bromides, and iodides; dialkyl sulfates, such as dimethyl, diethyl, dibutyl, and diamyl sulfates; long chain halides, such as decyl, lauryl, myristyl, and stearyl chlorides, bromides, and iodides; asthma halides, such as benzyl and phenethyl bromides; and others. Water or oil-soluble or dispersible products are thereby obtained.

As used herein, the term “phytosterol” refers to plant sterols and plant stanols. Plant sterols are naturally occurring cholesterol-like molecules found in all plants, with the highest concentrations occurring in vegetable oils. Plant stanols are hydrogenation compounds of the respective plant sterols. Phytosterols are natural components of common vegetable oils.

As used herein, the term “sensitivity” or “sensitive skin” refers to the degree of skin irritation or skin inflammation, as exemplified by parameters in suitable assays for measuring sensitivity, inflammation, irritation, and the like. One such assay is the Jordan-King assay, as set forth in Jordan, W. P. 1994, Jordan/King modification of the Draize Repeat Insult Patch Test, Clairol Study #94046, Test Dates Oct. 3, 1994-Nov. 11, 1994, the entire contents of which are hereby incorporated by reference.

As used herein, the phrase “skin protectant” refers to compositions that have the ability to repair interstitial lipid layers, provide lipid restoring, provide skin barrier restoration, and result in improvements in skin integrity so as to improve the appearance of skin.

Other terms as used herein are meant to be defined by their well-known meanings in the art.

### Skin Protectant Compositions

The present subject matter relates to various compositions suitable for use as a skin protectant. Accordingly, these compositions are suitable for topical application to skin of a mammal. These compositions preferably comprise five essential lipid components, namely a ceramide, a squalane, a phytosterol-containing liposome, a phospholipid-containing ingredient, and at least one triglyceride.

These five lipid components are preferably present in the instant compositions in specific, narrowly tailored amounts to maximize their effectiveness as skin protectants. In this regard, presently preferred compositions comprise about 0.001 to about 1.5% by weight of a ceramide, about 0.1 to about 5% by weight of a squalane, about 0.2 to about 5% by weight of a phytosterol-containing liposome, about 0.5 to about 5% by weight of a phospholipid-containing ingredient, about 8 to about 30% by weight of at least one triglyceride, and at least one dermatologically acceptable excipient. In a critical aspect, the presently preferred compositions are free of cholesterol.

The presently preferred topical compositions are specifically formulated to be capable of repairing or restoring a barrier abnormality in the stratum corneum, such as that seen in atopic dermatitis. The selection of the five classes of essential lipids conveys to the compositions their unique extended skin protection, restoration, and repair capabilities.

For example, the presence of a phytosterol-containing liposome in the presently preferred compositions is critical to providing these unexpected, advantageous capabilities. The vast majority of the previously known compositions contained cholesterol, or an animal-based sterol, rather than a phytosterol as an essential lipid component to fortify epidermal barriers. However, the use of a phytosterol rather than cholesterol conveys many unique advantages to the present compositions based on their inherent properties.

In this regard, phytosterols are typically incorporated in the basal membrane of the skin and can pass to the skin surface through the differentiation of skin cells. Accordingly, phytosterols usually provide an improved caring and protecting effect in skin cosmetics. The topical application of phytosterols also usually leads to an increased skin moisture level and to an increased lipid content. This improves the desquamation behavior of the skin and reduces erythemas which may be present. R. Wachter, Parf. Kosm., Vol. 75, p. 755 (1994) and R. Wachter, Cosm. Toil., Vol. 110, p. 72 (1995), each of which are incorporated herein by reference in their entirety, further demonstrate these advantageous properties of phytosterols.

Further, the presently preferred skin protectant compositions are uniquely formulated to provide an extended release of any and/or all of the five essential lipid ingredients contained therein. In this regard, the present compositions can be formulated to provide an extended release of an ingredient selected from the group consisting of the ceramide, squalane, phytosterol-containing liposome, phospholipid-containing ingredient, triglyceride, and combinations thereof. Accordingly, the present compositions can provide a release rate that is different from the normal release rate for any and/or all of these components. This extended release rate may result from the metabolizing or manipulation of the composition by the skin following topical administration, i.e. the compositions provide nutrients to promote normal skin barrier repair. This manipulation of the skin surface could result in improvements in the intracellular binding of cells through normal cell metabolic activity, leading to the end result of skin barrier repair through normal healing.

Further, due to this extended release profile, the present compositions are capable of providing extended beneficial effects on an area of skin to which they are applied. For example, the present compositions are capable of providing an extended moisturization in that they provide both a delayed moisturization of the skin (i.e. the moisturization commences later in time after administration than with administration of a previous product) as well as an extended moisturization of the skin (i.e. the moisturization continues for a longer period of time after administration than with administration of a previous product).

In addition, the present compositions are uniquely formulated so that they potentially minimize the amount of degradative components of the essential lipids contained therein. Accordingly, the present compositions can be specifically tailored to maintain high lipid purity and low levels of lipid degradates. The selection of specific excipients, as well as the preparation of an overall composition having a specific designated pH, can help to enable the present preferred formulations to maintain a unique purity and the absence of inherent degradates.

This lack of degradates is particularly important with respect to the ceramide component of the present compositions. The ability to maintain a high ceramide purity, and minimize the amount of ceramide degradates, permits the present compositions to have an improved adhesion to the skin. This improved adhesion, in turn, is at least partially responsible for the unexpectedly superior characteristics of the present compositions.

Additionally, the lack of significant amounts of degradative products in the present compositions makes them less irritating than topical skin protectant compositions previously known in the art. Accordingly, the present compositions are especially useful for application to sensitive or inflamed skin.

Further, the high purity level and low concentration of degradation products permit the present preferred compositions to have a longer shelf life when compared with other skin protectant products previously known in the art. In this regard, these compositions are able to maintain a low concentration of degradation product(s) of the essential components over an extended period of time. This advantageous property was heretofore unknown in previous skin protectant compositions.

The present compositions additionally preferable have a narrowly tailored pH of about 3 to about 9. These compositions preferably maintain this pH even though they typically do not contain a buffer. However, the present compositions are capable of containing certain buffer systems. Further, this pH profile is critical to the present compositions, as it permits these compositions to normalize the pH of skin to which they are applied to a predetermined optimal level for the promotion of an improved skin barrier function. Further, the present compositions tend to be creamy, stable, and anti-comedogenic.

Ceramides

The present compositions preferably comprise about 0.001 to about 1.5% by weight of a ceramide. The ceramide component of the present compositions is critical to the skin protectant features of these compositions, as ceramides are skin protecting agents that provide an excellent water barrier benefit. In this regard, the ceramide component functions to repair the skin barrier function generally, and to restore or repair a skin lipid barrier more specifically. This restoration or repair provides many of the unique skin treating properties and advantages of the present preferred compositions.

Ceramides can be extracted from brain tissue, nervous tissue, and other mammalian tissue, notably bovine brain, and human spleen tissue. A mixture termed “ceramides type III”, for example, is prepared by the action of phospholipase C on bovine brain sphingomyelin, and contains primarily stearic (18-carbon saturated) and nervonic acid moieties. Similarly, a mixture termed “ceramides type IV” is similar to ceramides type III except that it contains α-hydroxy acids rather than stearic and nervonic acids. All of these mixtures are commercially available from major chemical suppliers such as Sigma Chemical Company, St. Louis, Mo., U.S.A., and those which are not direct extracts from mammalian tissue are capable of being prepared by techniques described in the literature, such as Morrison, W. R., Biochem. Biophys. Acta. 176:537 (1969), and Carter, H. E., et al., J. Lipid Res. 2:228 (1961), the contents of which are hereby incorporated referenced in their entirety.

Non-limiting examples of ceramides useful in the present compositions include ceramide-I, -II, -III, -IV, -V, -VI, -VII, and mixtures thereof. Ceramide-III is particularly preferred in this regard. All known ceramides are expected to be effective in the present compositions. Other ceramides well known to those of skill in the art as useful in topical compositions are further contemplated as useful in the present compositions, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, January 1996, the contents of which are hereby incorporated by reference in their entirety.

Squalanes

The present compositions additionally preferably comprise about 0.1% to about 5% by weight of a squalane as an essential component. The squalane component of the present compositions is critical to their skin protectant effects, as squalanes are commonly known as vital oils effective as moisturizers that help enhance the skin's natural barrier function, protect the skin against the elements, and boost the skin's ability to retain moisture.

A preferred, non-limiting source of the squalane useful in the present compositions is shark liver oil, olive oil, rice bran, a derivative thereof, or a mixture thereof. Olive oil or a derivative thereof is particularly preferred in this regard. Other squalanes known to those of ordinary skill in the art, such as those described in the above resources, are further contemplated as useful in the present compositions.

Phytosterol-Containing Liposome

The present compositions additionally preferably comprise about 0.2 to about 5% by weight of a phytosterol-containing liposome. This phytosterol-containing liposome is critical to providing the unexpected, advantageous capabilities of the present compositions. It was previously unrecognized that the use of a plant-based phytosterol, rather than the common animal-based cholesterol, would provide topical compositions with improved abilities as a skin protectant composition.

In particular, the phytosterol component provides the present compositions with an improved caring and protecting effect in comparison with cholesterol containing formulations. The use of phytosterols also leads to an increased skin moisture level and to an increased lipid content. In this regard, the ability of the skin to readily metabolize phytosterol-containing liposomes permits the present compositions to increase intercellular adhesion in the skin after application to a mammal. This improves the desquamation behavior of the skin and reduces erythemas which may be present.

Further, the present compositions preferably contain an amount of the phytosterol-containing liposome effective to enhance the skin repair activity of the ceramide, described above. This represents an unexpected advantage over the previously known cholesterol-containing compositions.

Non-limiting examples of preferred phytosterol-containing liposomes useful in the present compositions are selected from the group consisting of shea butter, vegetable oil, tall oil, sesame oil, sunflower oil, sunflower seed oil, rice bran oil, cranberry seed oil, pumpkin seed oil, avocado wax, and mixtures thereof. Shea butter is a particularly preferred phytosterol-containing liposome in this regard. Other phytosterol-containing liposomes known to those of ordinary skill in the art, such as those described in the above resources, are further contemplated as useful in the present compositions.

The phytosterol-containing liposome is selected to contain at least one of any of the categories of phytosterols, for example 4-desmethylsterols, 4-monomethylsterols, and 4,4-dimethylsterols. In preferred embodiments, the phytosterol-containing liposome contains a sterol selected from the group consisting of β-sitosterol, β-sitostanol, compesterol, sigmasterol, and mixtures thereof.

Phospholipid-Containing Ingredient

The present compositions additionally preferably comprise about 0.5% to about 5% by weight of a phospholipid-containing ingredient as an essential component. The phospholipid stabilizes the oil components present in these compositions. In this regard, the phospholipid spontaneously forms closed fluid-filled vesicles when mixed in suitable concentrations with water.

A preferred, non-limiting phospholipid-containing ingredient useful in the present compositions is hydrogenated lecithin. Other phospholipid-containing ingredients known to those of ordinary skill in the art, such as those described in the above resources, are further contemplated as useful in the present compositions.

Triglycerides

The present compositions additionally preferably comprise about 8% to about 30% by weight of at least one triglyceride as an essential component. A preferred, non-limiting triglyceride useful in the present compositions is caprylic/capric triglyceride. Other triglycerides known to those of ordinary skill in the art, such as those described in the above resources, are further contemplated as useful in the present compositions.

Essential Fatty Acids

The present skin protectant compositions may additionally comprise at least one essential fatty acid. Several non-limiting examples of such essential fatty acids useful in the present compositions include linoleic acid, linolenic acid, oleic acid, columbinic acid, arachidic acid, arachidonic acid, lignoceric acid, nervonic acid, eicosapentanoic acid, palmitic acid, stearic acid, and mixtures thereof. Other essential fatty acids known to those of ordinary skill in the art, such as those described in the above resources, are further contemplated as useful in the present compositions.

The at least one essential fatty acid can be introduced into the present compositions from a variety of sources. In preferred embodiments, the at least one essential fatty acid is provided in the compositions as an oil. Non-limiting examples of oils useful in this regard include flaxseed oil, hempseed oil, pumpkin seed oil, canola oil, soybean oil, wheat germ oil, olive oil, grapeseed oil, borage oil, evening primrose oil, black currant seed oil, chestnut oil, corn oil, safflower oil, sunflower oil, sunflower seed oil, cottonseed oil, peanut oil, sesame oil, vegetable oil, and mixtures thereof. Other essential fatty acid-containing oils, as well as other fatty acid-containing sources. known to those of ordinary skill in the art, such as those described in the above resources, are further contemplated as useful in the present compositions.

Dermatologically Acceptable Excipients

The present skin protectant compositions additionally comprise at least one dermatologically acceptable excipient commonly known to those of ordinary skill in the art as useful in topical compositions. Several non-limiting examples of such additional excipients include an emollient, a moisturizer, a preservative, a gelling agent, a colorant or pigment, and mixtures thereof. Other common dermatologically acceptable excipients known to those of ordinary skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, January 1996, the contents of which are hereby incorporated by reference in their entirety, may be further present in the instant compositions.

Non-limiting examples of specific emollients useful in the present skin protectant compositions include vegetable oils, coconut oil, palm glycerides, olea europaea, extracts thereof, derivatives thereof, and mixtures thereof. Other emollients well known to those of skill in the art, such as those described in the above resources, are further contemplated as useful in the present compositions.

Non-limiting examples of specific moisturizers useful in the present skin protectant compositions include glycerin, pentylene glycol, butylene glycol, polyethylene glycol, sodium pyrrolidone carboxylate, α-hydroxy acids, β-hydroxy acids, polyhydric alcohols, ethoxylated and propoxylated polyols, polyols, polysaccharides, panthenol, hexylene glycol, propylene glycol, dipropylene glycol, sorbitol and mixtures thereof. In preferred embodiments, the moisturizer will have no more than an eight carbon chain length. Other moisturizers known to those of ordinary skill in the art, such as those described in the above resources, are further contemplated as useful in the present compositions.

Non-limiting examples of specific preservatives useful in the present topical compositions include propylene glycol, glycerol, butylene glycol, pentylene glycol, hexylene glycol, sorbitol, and mixtures thereof. Pentylene glycol is particularly preferred in this regard. Other preservatives known to those of ordinary skill in the art, such as those described in the above resources, are further contemplated as useful in the present compositions.

Non-limiting examples of specific gelling agents useful in the present compositions include various cellulose agents, hydroxyethylcellulose, xanthan gum, sodium carbomer, carbomer, and mixtures thereof. Other suitable gelling agents which may be useful in the present compositions include aqueous gelling agents, such as neutral, anionic, and cationic polymers, and mixtures thereof. Exemplary polymers which may be useful in the instant compositions include carboxy vinyl polymers, such as carboxypolymethylene. A preferred gelling agent is a Carbopol® polymer such as is available from Noveon Inc., Cleveland, Ohio. Carbopol® polymers are high molecular weight, crosslinked, acrylic acid-based polymers. Carbopol® homopolymers are polymers of acrylic acid crosslinked with allyl sucrose or allylpentaerythritol. Carbopol® copolymers are polymers of acrylic acid, modified by long chain (C10-C30) alkyl acrylates, and crosslinked with allyl-pentaerythritol.

Other suitable gelling agents include cellulosic polymers, such as gum arabic, gum tragacanth, locust bean gum, guar gum, xanthan gum, cellulose gum, methylcellulose, hydroxyethylcellulose, hydroxypropylcellulose, and hydroxypropylmethylcellulose.

Further, any common gelling agents known to those of ordinary skill in the art, such as those described in the above resources, are further contemplated as useful in the present compositions.

The topical compositions contemplated herein may be in a lotion, cream, ointment, shampoo, gel, paste, skin cleanser, aerosol, or other dermatologically acceptable topical dosage form. Other cosmetic treatment compositions known to those skilled in the art, including liquids and balms, are additionally contemplated as falling within the scope of the present subject matter.

Creams useful herein are easily applied and vanish when rubbed into the skin. Lotions useful herein include suspensions of powdered material in a water or alcohol base (e.g., calamine). Convenient to apply, lotions are also cool and help to dry acute inflammatory and exudative lesions.

Ointments which are useful herein are oleaginous and contain little if any water; feel greasy but are generally well tolerated; and are best used to lubricate, especially if applied over hydrated skin. These ointments are preferred for lesions with thick crusts, lichenification, or heaped-up scales and may be less irritating than cream formulations for some eroded or open lesions (e.g., stasis ulcers). Drugs in ointments are often more potent than in creams.

### Combination Therapy

The subject matter described herein further contemplates administering the above-described skin protectant compositions in combination with a pharmacologically active agent. This pharmacologically active agent is administered topically or orally either concomitantly or sequentially with the above described skin protectant compositions. Accordingly, the pharmacologically active agent is administered with the skin protectant compositions either in adjunctive or co-therapy. That is, the pharmacologically active agent can either be administered as a component of the skin protectant composition or as part of a second, separate composition. This second, separate composition can be either an oral or a topical composition.

In preferred embodiments, the present skin protectant composition enhances the effectiveness of the pharmacologically active agent. This enhanced effectiveness may result from an improved solubility profile of the pharmacologically active agent.

Exemplary pharmacologically active agents that are effectively used in combination with the present skin protectant compositions include, but are not limited to, a local anesthetic, a steroid, an anti-inflammatory agent, an antibiotic, an antiviral agent, an antifungal, an antihistamine, an antipruritic, an antineoplastic agent, natural and synthetic vitamins and analogs, a cytotoxic agent, an anti-infective agent, an immune-modulating agent, a sunscreen, a sunblock, an agent useful in treating skin diseases, and mixtures thereof. A local anesthetic is particularly preferred in this regard.

Furthermore, the formulation may be used with adjunct therapies and treatments, such as pre-washing with common soaps, and mild detergents.

### Composition Delivery Systems

In another preferred embodiment, the present subject matter additionally provides unique delivery systems designed for topical application to skin of a mammal. In this regard, the present delivery systems contain therapeutically effective amounts of the hereinbefore described skin protectant composition and a carrier for this composition. In a critical aspect, all of the delivery system, skin protectant composition, and carrier are free of cholesterol.

The present delivery systems are selected to provide an extended release of each of the five essential lipid components of the present skin protectant compositions, namely the ceramide, squalane, phytosterol-containing liposome, phospholipid-containing ingredient, and at least one triglyceride. In this regard, only those delivery systems capable of delivering the present skin protectant compositions to the skin of a mammal in such a way as optimize, enhance, and take advantage of the unique extended release characteristics of the present compositions are contemplated for use herein. In a preferred embodiment, the present delivery system is a device for administering these compositions to the skin of a mammal.

Non-limiting examples of preferable suitable carriers used for the present delivery systems include a transdermal patch, a band-aid, a gauze bandage, a mask, and combinations thereof.

In an alternative preferred embodiment, the carrier for the present delivery systems is an applicator. Non-limiting examples of applicators useful in this regard include a pledget, a pad, a sponge, a delivery tube, a delivery spout, and combinations thereof.

### Methods of Skin Restoration, Repair, and Treatment

The skin protectant compositions described herein are preferably used in methods for restoring or repairing a skin lipid barrier of a mammal. The present skin protectant compositions can further fortify the barrier to prevent its disruption due to environmental insults. In this regard, the present methods contemplate topically applying to skin of a mammal in need thereof a therapeutically effective amount of the present preferred skin protectant compositions. Once topically applied to the mammal, these skin protectant compositions increase intercellular adhesion in the skin of the mammal. This increased intercellular adhesion results in the restoration and/or repair of the skin lipid barrier, as well as moisturization of the skin. Further, the increased intercellular adhesion is a direct response to the skin's metabolizing the phytosterol-containing liposome in the present compositions.

The repair of the skin lipid barrier by the present preferred compositions improves the skin barrier function and conveys numerous additional therapeutic effects to a mammal to which the compositions are applied. For example, this skin lipid barrier repair can further enhance the repair of the skin to which the compositions are applied, increase the interstitial oil content of the skin, improve the integrity of the skin's interstitial lipid layer, treat dry skin, provide a reduced incidence of atopic dermatitis in a mammal predisposed to atopic dermatitis, and reduce the occurrence of further skin barrier malfunctions. In this regard, the increased interstitial oil content of the skin and the improved integrity of the skin's interstitial lipid layer have a direct result on the enhanced skin repair. Accordingly, the present skin protectant compositions are unexpectedly useful in methods of treating any of these skin areas or skin conditions. Measuring the transepidermal water loss (TEWL) through the skin barrier can be a sensitive and reliable way to measure the amount and extent of the barrier repair.

The improved skin barrier function may be a result of the unique pH characteristics of the present compositions. The specific, narrow pH of the present compositions, i.e. a pH of about 3 to about 9, has a significant impact upon application to the skin. In particular, the present compositions have the unique ability to normalize the pH of the skin to a predetermined optimal skin pH. This normalized skin pH results in an improved skin barrier function.

In addition to and concurrently with the skin repair, the increased intercellular adhesion resulting from administration of the present preferred compositions can further reduce manifestations of dry skin while enhancing the skin repair. This reduction of dry skin manifestations is optimally achieved by daily topically applying these compositions to the skin of a mammal. The present preferred compositions are superior to those compositions presently available for the reduction of dry skin, and thus for the moisturization of the skin, due to their extended release characteristics. Accordingly, these compositions are capable of providing both a delayed moisturization and an extended moisturization of the skin.

The present preferred skin protectant compositions can further be effective in treating a variety of skin conditions characterized by sensitive skin. Typically, sensitive skin can be the result of a reduced or underdeveloped skin barrier function. For example, children's skin is often very sensitive as the skin barrier function has not yet had sufficient time to fully develop, i.e. it is underdeveloped. Similarly, the skin barrier function of skin of patients aged 55 years and older is often negatively affected by years of normal wear and tear, resulting in a reduced skin barrier function. These less than optimal skin barrier functions often result in especially sensitive skin since the skin's barrier and ability to withstand outside irritating agents is reduced.

Accordingly, the present preferred compositions can be especially effective in treating skin conditions associated with and/or caused by sensitive skin. The extended release attributes of these skin protectant compositions permit the release of each of the essential lipid components to the skin in such a manner and over such a time period so as to sufficiently treat sensitive skin without causing further irritation of the sensitive skin. This is a distinct advantage over other skin protectant compositions previously known in the art. In this regard, the present preferred compositions can be topically applied to sensitive skin areas, irritated skin areas, or inflamed skin areas.

In preferred embodiments, non-limiting examples of the skin conditions treatable with the present skin protectant compositions include those selected from the group consisting of atopic dermatitis, pruritis, itching, eczema, ichthyosis, psoriasis, seborrheic dermatitis, eczematous dermatitis, ulcers and erosions due to cutaneous trauma, epidermolysis bullosa, cutaneous changes of intrinsic or extrinsic aging, and a combination thereof. Atopic dermatitis is particularly preferred in this regard. In an especially preferred embodiment, the present subject matter further contemplates reducing the incidence of further occurrences of these skin conditions, in addition to the initial treatment.

In further preferred aspects, the mammal treatable with the present skin protectant compositions is a human. Particularly preferred humans in this regard are human children. In especially preferred embodiments, the human children treated have an age of up to and including 6 years old. In a most preferred embodiment, the human children have an age of up to and including 2 years old.

In an alternative particularly preferred embodiment, the preferred humans treated according to the present subject matter are humans of at least 55 years old. Additional particularly preferred embodiments of the present subject matter contemplate methods of treating skin conditions in females.

In another preferred embodiment, the topical application of the present compositions reduces the redness, flushing, and blushing associated with rosacea. The treatment for rosacea described herein can also be effective in treating other skin disorders or conditions associated with, or commonly further occurring in skin having, a reduced, underdeveloped, and/or otherwise damaged skin barrier function.

### Process for Preparing

The present subject matter further relates to a process for preparing a composition suitable for topical administration comprising an oil and water emulsion. This unique process comprises the steps of:

1) preparing an aqueous phase comprising about 5 to about 20% of the overall weight of the composition of at least one moisturizer, a first gelling agent, and about 10 to about 60% of the overall weight of the composition of water,

2) cooling said aqueous phase to a temperature of about 40 to about 50° C.;

3) preparing an oil phase comprising about 0.1 to about 5% by weight of the overall weight of the composition of a squalane, about 0.1 to about 5% by weight of the overall weight of the composition of a phytosterol-containing liposome, about 5 to about 25% of the overall weight of the composition of at least one triglyceride, and a second gelling agent;

4) adding said water phase to said aqueous phase while stirring at a temperature of about 40 to about 50° C. to obtain an emulsion;

5) cooling said emulsion to a temperature of about 25 to about 35° C.;

6) adding a ceramide and a phospholipid-containing ingredient to said emulsion; and

7) recovering a topical composition.

In a preferred embodiment, the aqueous phase is prepared according to said process step 1) by first mixing the water of this process step and the at least one moisturizer before addition of the first gelling agent. In a preferred embodiment, the first gelling agent is added to the aqueous phase while heating the aqueous phase to a temperature of about 50 to about 70° C. under fast stirring. In a particularly preferred embodiment, the first gelling agent is hydroxyethylcellulose.

In another preferred embodiment, the aqueous phase is mixed after the first gelling agent is added until the gelling agent has swelled completely and the aqueous phase is clear.

In a further preferred embodiment, the oil phase is prepared according to said process step 3) by first mixing the squalane, the phytosterol-containing liposome, and the at least one triglyceride before the second gelling agent is added. In this regard, the second gelling agent is preferably added to the oil phase while heating the oil phase to a temperature of about 40 to about 50° C. under slow stirring. In a particularly preferred embodiment, the second gelling agent is selected from the group consisting of xanthan gum, carbomer, sodium carbomer, and mixtures thereof.

In an alternative embodiment of the present processes, the composition is prepared by providing a change in flow of an aqueous phase and an oil phase comprising a squalane, a phytosterol-containing liposome, and at least one triglyceride to provide an oil-in-water emulsion; adding a ceramide and a phospholipid-containing ingredient to the emulsion; and recovering a topical composition. This process does not require the presence of an emulsifier to form the emulsion. In a particularly preferred aspect, the change in flow is caused by a change in pressure. This change in pressure is preferably a change from atmospheric pressure to a pressure of about 5,000-25,000 psig. In a particularly preferred embodiment, the change in pressure is a change from atmospheric pressure to a pressure of about 10,000 psig. Accordingly, the present processes have a reduced dependence on emulsifiers in forming the present emulsion compositions, in particular polyethylene glycol emulsifiers.

According to this alternative process, the aqueous phase comprises about 5 to about 20% of the overall weight of the composition of at least one moisturizer, a first gelling agent, and about 10 to about 60% of the overall weight of the composition of water. Similarly, the oil phase comprises about 0.1 to about 5% by weight of the overall weight of the composition of the squalane, about 0.1 to about 5% by weight of the overall weight of the composition of the phytosterol-containing liposome, about 5 to about 25% of the overall weight of the composition of the at least one triglyceride, and a second gelling agent.

In an alternative preferred embodiment of the present processes, the emulsion is prepared through the use of ultrasonic waves, and does not require the presence of an emulsifier to form the emulsion.

The present processes preferably form compositions comprising an emulsion having an oil phase and an aqueous phase. Non-limiting examples of specific types of emulsions that can be made according to this process include an oil-in-water emulsion, a water-in-oil emulsion, an oil-in-water-in-oil emulsion, and a water-in-oil-in-water emulsion. The formation of a specific type of emulsion will depend on the specific ingredients used in the process. In a preferred embodiment, the process will form compositions that are oil-in-water emulsions.

Further, these particular preparation processes are non-limiting examples of possible processes that can be used to prepare the present compositions. Other processes capable of preparing these compositions are further contemplated herein. Further, the individual phases of the present compositions (for example aqueous and oil phases) can be prepared sequentially in any order or concurrently; it is not a necessary aspect of the present processes that the aqueous phase be prepared before the oil phase is prepared. Additionally, the present compositions can be prepared according to either a batch process or continuously.

Further contemplated as within the scope of the present subject matter are compositions produced according to the above-described processes. If produced according to these processes, these compositions exhibit chemical and physical stability suitable for topical administration.

The compositions produced according to these processes can be placed in a suitable containment vessel comprising a product contact surface composed of a material selected from the group consisting of glass, plastic, steel, stainless steel, aluminum, Teflon, polymeric structure, ceramic structure, alloys, and mixtures thereof. These containment vessels are used to facilitate manufacturing, handling, processing, packaging, storage, and administration of said composition. Preferred containment vessels in this regard can be selected from the group consisting of plastic tubes, bottles, metal tubes, and any combination thereof.

### Routes of Administration/Dosage

To be effective, the route of administration for the compositions used in the present preferred methods must readily affect the target skin areas. Effective results in most cases are achieved by topical application of a thin layer over the affected area, or the area where one seeks to achieve the desired effect. Effective results can be achieved with application rates from one application every two or three days to four or more applications per day.

Appropriate dosage levels are well known to those of ordinary skill in the art and are selected to maximize the treatment of the above skin conditions. Dosage levels on the order of about 0.001 mg to about 5,000 mg per kilogram body weight of the essential lipids present in the skin protectant compositions are known to be useful in the methods described herein. Typically, this effective amount of the five essential lipids will generally comprise from about 0.001 mg to about 100 mg per kilogram of patient body weight per day.

The specific dose level for any particular patient will vary depending upon a variety of factors, including the activity of the specific lipids employed; the age, body weight, general health, sex and diet of the patient; the time of administration; the rate of excretion; possible drug combinations; the severity of the particular condition being treated; and the form of administration. Typically, in vitro dosage-effect results can provide useful guidance on the proper doses for patient administration. Studies in animal models are also helpful. The considerations for determining the proper dose levels are well known in the art and are incorporated herein for the present subject matter.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time are well known in the art.

Lessening exposure by once-daily administration affects multiple pharmacokinetic parameters and provides the initial mechanism for avoiding skin irritation and inflammation and the other toxicity issues discussed herein.

The optimal formulations will be determined by one skilled in the art depending upon considerations such as the particular lipid combination and the desired dosage. See, for example, “Remington's Pharmaceutical Sciences”, 18th ed. (1990, Mack Publishing Co., Easton, Pa. 18042), pp. 1435-1712, the disclosure of which is hereby incorporated by reference. Such formulations may influence the physical state, stability, rate of in vivo release, and rate of in vivo clearance of the essential lipids.

Single dosage kits and packages containing once per day amount of composition may be prepared. Single dose, unit dose, and once-daily disposable containers of the present compositions are contemplated as within the scope of the present subject matter.

The present compositions may be formulated for storage in a substantially non-reactive laminated package to enhance stability of the package. This method of storage provides enhanced package stability in comparison with other, paper-based packages.

The amount of composition per single packet may range be from about 0.1 mL to about 20.0 mL, preferably between about 0.5 and about 5.0 mL, more preferably between about 1 and about 3 mL.

In particular, the ability to formulate compositions capable of long term storage, without pre-mixing or compounding requirements prior to application, are also contemplated. Specifically, the present preferred compositions remain unexpectedly stable in storage for periods including between about 3 and about 18 months, preferably between about 3 and about 15 months, more preferably between about 3 and about 12 months, and alternately any time period between about 6 and about 18 months.

### EXAMPLES

The following examples are illustrative of the present subject matter and are not intended to be limitations thereon. All polymer molecular weights are mean average molecular weights. All percentages are based on the percent by weight of the final delivery system or formulation prepared unless otherwise indicated and all totals equal 100% by weight.

### Example 1

The following example illustrates a manufacturing formula for a cream composition of the present subject matter:

| | % W/W |
|---|---|
| Water | 56.8475 |
| Caprylic/Capric Triglyceride | 22.4 |
| Glycerin | 8.75 |
| Pentylene Glycol | 4.75 |
| Coconut Oil | 3.5 |
| Hydrogenated Lecithin | 1.5 |
| Shea Butter | 1.35 |
| Hydroxyethylcellulose | 0.35 |
| Squalane | 0.25 |
| Carbomer | 0.1 |
| Sodium Carbomer | 0.1 |
| Xanthan Gum | 0.1 |
| Ceramide 3 | 0.0025 |
| | 100.0% |

This final composition can be prepared as follows:

1. An aqueous phase is prepared by mixing the pentylene glycol, glycerin, and purified water. The hydroxyethylcellulose (HEC) is then added with slow homogenizing. Once all the HEC has been added the homogenizer is switched off. This mixture is then stirred fast while heating to a temperature of 60±3° C. and avoiding foaming. The stirring is continued for about 20 minutes, or until the HEC swells completely and the aqueous phase is clear. The aqueous phase is then cooled to a temperature of 40±3° C., and homogenized while cooling.

2. An oil phase is prepared by mixing the Caprylic/Capric Triglyceride, Squalane, Shea Butter, Coconut Oil, and Xanthan Gum while heating to a temperature of 42±3° C. The Carbomer and Sodium Carbomer are then added under slow homogenization until dispersed. The mixture is then heated to 42±3° C.

3. While stirring, the aqueous phase is quickly added to the oil phase under vacuum. This mixture is stirred fast with fast homogenization and recirculation for about 35 minutes, while maintaining the temperate at 40±3° C. to form an emulsion. The emulsion is then cooled to about 30° C. while maintaining the stirring at a maximum 45 rpm fast homogenization.

4. The Ceramide-3 and the Hydrogenated Lecithin are then added to the emulsion while stirring at about 30 rpm. While stirring at about 30 rpm, the emulsion is homogenized at about 3000 rpm for about 55 minutes at a temperature of not more than 34° C. Once all large solid agglomerates have been removed, the emulsion is cooled to 25-27° C. while stirring at about 30 rpm.

### Example 2

The following example illustrates a manufacturing formula for a lotion composition of the present subject matter:

| | % W/W |
|---|---|
| Lipid Concentrate | 15.0 |
| Palm Glycerides | 1.4 |
| Caprylic/Capric Triglyceride | 11.0 |
| Shea Butter | 0.6 |
| Coconut Oil | 2.0 |
| Olea Europaea | 1.0 |
| Caprylyl Glycol | 0.25 |
| Carbomer | 0.05 |
| Xanthan Gum | 0.2 |
| Sodium Carbomer | 0.05 |
| Purified Water | 55.85 |
| Pentylene Glycol | 4.25 |
| Hydroxyethylcellulose | 0.35 |
| Glycerol | 8.0 |
| | 100.0% |

This lotion is prepared according to the process described above for Example 1.

### Example 3

A patient is suffering from atopic dermatitis. A skin protectant composition of the present subject matter is topically administered to the patient. It would be expected that the patient would improve his/her condition or recover.

### Example 4

A patient is suffering from dry skin. A skin protectant composition of the present subject matter is topically administered to the patient. It would be expected that the patient would improve his/her condition or recover.

### Example 5

A patient is suffering from a damaged skin lipid barrier. A skin protectant composition of the present subject matter is topically administered to the patient. It would be expected that the patient would improve his/her condition or recover.

The present subject matter being thus described, it will be apparent that the same may be modified or varied in many ways. Such modifications and variations are not to be regarded as a departure from the spirit and scope of the present subject matter, and all such modifications and variations are intended to be included within the scope of the following claims.

We claim:

1. A delivery system for topical application to skin of a mammal comprising:
a therapeutically effective amount of a skin protectant composition comprising:
a) a ceramide,
b) a squalane,
c) a phytosterol-containing liposome,
d) a phospholipid-containing ingredient,
e) at least one triglyceride, and
f) at least one dermatologically acceptable excipient; and
a carrier for said skin protectant composition, wherein said skin protectant composition is free of cholesterol.

2. The delivery system of claim 1, wherein said carrier is selected from the group consisting of a transdermal patch, a band-aid, a gauze bandage, and a mask.

3. The delivery system of claim 1, wherein said carrier is an applicator selected from the group consisting of a pledget, a pad, a sponge, a delivery tube, a delivery spout, and combinations thereof.

4. The delivery system of claim 1, wherein said skin protectant composition is in a lotion, cream, ointment, shampoo, gel, paste, skin cleanser, aerosol, or other dermatologically acceptable topical dosage form.

5. The delivery system of claim 1, wherein said skin protectant composition comprises from about 0.001 to about 1.5% by weight of said ceramide, from about 0.1 to about 5% by weight of said squalane, from about 0.2 to about 5% by weight of said phytosterol-containing liposome, from about 0.5 to about 5% by weight of said phospholipid-containing ingredient, and from about 8 to about 30% by weight of said at least one triglyceride.

6. The delivery system of claim 1, further comprising a local anesthetic.

7. A method for restoring or repairing a skin lipid barrier of a mammal comprising:
topically applying to skin of a mammal in need thereof a therapeutically effective amount of a composition comprising:
a) a ceramide;
b) a squalane;
c) a phytosterol-containing liposome;
d) a phospholipid-containing ingredient;
e) at least one triglyceride; and
f) at least one dermatologically acceptable excipient,
wherein said composition increases intercellular adhesion in said skin to restore or repair said skin lipid barrier and enhance moisturization of said skin, and wherein said composition is free of cholesterol.

8. The method of claim 7, wherein said increased intercellular adhesion results from said skin's metabolizing said phytosterol-containing liposome.

9. The method of claim 7, wherein said composition provides an extended release of an ingredient selected from the group consisting of said ceramide, squalane, phytosterol-containing liposome, phospholipid-containing ingredient, triglyceride, and combinations thereof.

10. The method of claim 9, wherein said extended release provides an extended or delayed moisturization of said skin.

11. The method of claim 9, wherein said extended release allows said composition to further treat a skin condition in a mammal having sensitive skin.

12. The method of claim 11, wherein said skin condition is selected from the group consisting of atopic dermatitis, pruritis, itching, eczema, ichthyosis, psoriasis, seborrheic dermatitis, eczematous dermatitis, ulcers and erosions due to cutaneous trauma, epidermolysis bullosa, cutaneous changes of intrinsic or extrinsic aging, dry skin, and a combination thereof.

13. The method of claim 11, wherein said method additionally reduces the incidence of further occurrences of said skin condition.

14. The method of claim 7, wherein said repair of said skin lipid barrier improves integrity of the skin's interstitial lipid layer.

15. The method of claim 7, wherein said method additionally reduces the occurrence of further skin barrier malfunctions.

16. The method of claim 7, wherein said phytosterol-containing liposome is shea butter, said ceramide is ceramide-3, said triglyceride is caprylic/capric triglyceride, said squalane is olive oil or a derivative thereof, and said phospholipid-containing ingredient is hydrogenated lecithin.

17. The method of claim 7, wherein said dermatologically acceptable excipient is selected from the group consisting of an emollient, a moisturizer, a preservative, a gelling agent, a colorant or pigment, and mixtures thereof.

18. The method of claim 7, wherein said composition further comprises at least one essential fatty acid.

19. The method of claim 7, wherein said composition is administered in combination with a pharmacologically active agent.

20. The method of claim 19, wherein said pharmacologically active agent is a local anesthetic.

21. The method of claim 19, wherein said pharmacologically active agent is administered either concomitantly or sequentially with said composition.

22. The method of claim 19, wherein said composition enhances the effectiveness of the pharmacologically active agent.

23. The method of claim 7, wherein said composition normalizes the pH of the skin resulting in an improved skin barrier function.

24. The method of claim 23, wherein said composition does not contain a buffer.

25. A process for manufacturing a composition suitable for topical administration comprising an oil-in-water emulsion, said process comprising:
1) providing a change in flow of an aqueous phase and an oil phase comprising a squalane, a phytosterol-containing liposome, and at least one triglyceride to provide an oil-in-water emulsion;
2) adding a ceramide and a phospholipid-containing ingredient to said emulsion; and
3) recovering a topical composition.

26. The process of claim 25, wherein said aqueous phase comprises about 5 to about 20% of the overall weight of the composition of at least one moisturizer, a first gelling agent, and about 10 to about 60% of the overall weight of the composition of water,
and wherein said oil phase comprises about 0.1 to about 5% by weight of the overall weight of the composition of the squalane, about 0.1 to about 5% by weight of the overall weight of the composition of the phytosterol-containing liposome, about 5 to about 25% of the overall weight of the composition of the at least one triglyceride, and a second gelling agent.

27. The process of claim 25, wherein said change in flow is caused by a change in pressure.

28. The process of claim 27, wherein said change in pressure is a change from atmospheric pressure to a pressure of about 5,000-25,000 psig.

29. The process of claim 25, wherein said process further comprises:
1) preparing said aqueous phase;
2) cooling said aqueous phase to a temperature of about 40 to about 50° C.;
3) preparing said oil phase;
4) adding said water phase to said aqueous phase while stirring at a temperature of about 40 to about 50° C. to obtain an emulsion;
5) cooling said emulsion to a temperature of about 25 to about 35° C.;
6) adding a ceramide and a phospholipid-containing ingredient to said emulsion; and
7) recovering a topical composition.

30. The process of claim 29, wherein said process reduces dependence on emulsifiers in forming said emulsion.
